(19) United States
(12) Patent Application Publication
(10) Pub. No.: US 2023/0054841 A1
(43) Pub. Date: Feb. 23, 2023

(54) Methods for Directed Exaptation

(71) Applicant: Massachusetts Institute of Technology, Cambridge, MA (US)

(72) Inventors: Andreas Mershin, Cambridge, MA (US); Shannon Leigh Johnson, Cambridge, MA (US)

(73) Assignee: Massachusetts Institute of Technology, Cambridge, MA (US)

(21) Appl. No.: 17/870,807

(22) Filed: Jul. 21, 2022

Related U.S. Application Data
(63) Continuation of application No. 16/133,600, filed on Sep. 17, 2018, now abandoned.
(60) Provisional application No. 62/559,550, filed on Sep. 16, 2017.

(51) Int. Cl. C12N 15/01; C12Q 1/10; C12N 1/20; A61K 35/74

(52) U.S. Cl. CPC C12N 15/01; C12Q 1/10; C12N 1/20; A61K 35/74; C12N 1/205; C12R 2001/19

(57) ABSTRACT

A method for directed exaptation includes dividing an original microorganism monoculture into subcultures that are subjected to different exaptation agents to obtain diversified substrains. At least one of the exaptation agents is selected to favor survival of sub strains exhibiting desired traits. The steps of dividing and subjecting may be iterated using at least some of the diversified substrains. Performance of diversified substrains is assessed and those that meet performance criteria for at least one desired trait are selected. Exaptation agents may include mutagenesis agents, training, horizontal gene transfer opportunities, and stressors. Substrains may be co-incubated with other living or dead microorganisms known to be preferentially adapted to have the desired trait. Diversified substrains may be combined into a multiculture microorganism population, to which microorganisms from the original monoculture may be added. The method may be used to create a treatment for a Multiple-Antibiotic Resistant Infection, preferably including a kill switch.

### RELATED APPLICATIONS

This application is a continuation of U.S. patent application Ser. No. 16/133,600, filed Sep. 17, 2018, which claims the benefit of U.S. Provisional Application Ser. No. 62/559,550, filed Sep. 16, 2017, the entire disclosures of which are herein incorporated by reference.

### FIELD OF THE TECHNOLOGY

The present invention relates to directed evolution, genome engineering, and synthetic biology and, in particular, to methodologies for, and clinical applications of, exaptation.

### BACKGROUND

In the field of directed evolution there are at least two distinct concepts: Adaptation and Exaptation. Adaptation is generally defined as an enhancement of an existent trait, resulting in an increased fitness of an organism or population, such as, for instance, the lengthening of a giraffe's neck. Exaptation is here defined as an evolving system's capacity to discover new functions by acquiring and deploying one or more adaptive traits in novel ways, such as the case of feathers for flight [Barve, Aditya and Wagner, Andreas, “A latent capacity for evolutionary innovation through exaptation in metabolic systems”, Nature 500, 203-206, Aug. 8, 2013].

The Weizmann Evolthon Genome Engineering Challenge competition was a lab evolution challenge for the community of scientists interested in microbial evolution, focused on finding and comparing new strategies for microorganisms' lab evolution toward competing against each other under a given challenge in a laboratory setting. Evolthon took place as a part of the “Genome Evolution” meeting at the Weizmann institute of Science on 1-3 Nov. 2016. The present invention was submitted as an entry into the Evolthon 2016 competition.

### SUMMARY

The present invention relates to methods for directed exaptation using multicultures of bacterial populations and methods for rapid prototyping of such populations that outcompete wild type populations, including multicultures as treatment for specific Multiple-Antibiotic Resistant Infections (MARI). In example embodiments, methods according to the invention may be applied to the rapid generation of genetically and phenotypically diverse microorganism populations starting from a monoculture, including, but not limited to, microorganisms capable of leveraging horizontal gene transfer when presented with stressors, such as, but not limited to, E. coli. Methods according to the invention may include automated or user-supervised feedback to control and discover (sets of) mutations of interest to biomanufacturing of molecular products, such as novel or extant therapeutics. End-use applications include a novel genome engineering and genome design and evolution research methodology, and the production of mono- and multi-cultures customized to safely outcompete multiple-antibiotic resistant infections (MARI).

The invention provides a process for directing production of varying types of monoculture strains, optionally pooled into multicultures, that maximize the probability for exaptation to occur at the individual or population level and may be used to rapidly screen for optimal performance to arbitrary challenges, such as, for instance, the rapid creation of a multi-mutation bearing mono- or multiculture developed to maximize performance to a specific challenge, such as, for example, growth at suboptimal temperature or biomanufacturing yield, or creation of a patient-specific anti-MARI multiculture incorporating a safety “kill switch”.

In one aspect, the invention is a method for directed exaptation of microorganisms that includes the steps of dividing an original population comprising a monoculture of microorganisms into a plurality of subcultures and subjecting the plurality of subcultures to a plurality of exaptation agents in order to obtain diversified substrains, wherein at least some of the subcultures are subjected to different exaptation agents than other subcultures and wherein at least one of the exaptation agents is selected to favor survival of substrains exhibiting one or more desired traits. The steps of dividing and subjecting may be optionally iterated, using at least some of the diversified sub strains in place of the original population, until a desired level of diversification of substrain traits is achieved. Performance of each of the diversified substrains is assessed with respect to at least one of the desired traits, and those of the diversified substrains that meet predetermined performance criteria for at least one of the desired traits are selected. The exaptation agents may include at least mutagenesis agents, training, horizontal gene transfer opportunities, and stressors. At least some of the substrains may be co-incubated with at least one other living or dead microorganism, wherein the other microorganism is known to be preferentially adapted to have the desired trait. At least some of the diversified substrains may be combined into a multiculture microorganism population, to which at least some microorganisms from the original monoculture may optionally be added. At least some of the subcultures may also be subjected to at least one adaptation-evoking agent. The invention incudes any multiculture microoganism population and any microorganism substrain population created according to the method.

In another aspect, the invention is a multiculture microorganism population specifically exapted to meet a specific challenge condition, the multiculture microorganism population comprising a plurality of microorganism substrains derived from an original monoculture, wherein the individual substrains have been individually and collectively subjected to exaptation agents in order to induce changes within the substrains that cause the multiculture to meet the challenge condition. The multiculture microorganism population may include at least some microorganisms from the original monoculture. At least some of the substrains may have been co-incubated with at least one other living or dead microorganism, wherein the other microorganism is known to be preferentially adapted to have the desired trait.

In yet another aspect, the invention is a method for producing a treatment for a Multiple-Antibiotic Resistant Infection that includes the steps of creating an antiMARI microorganism by the steps of dividing an original population comprising a monoculture of a plurality of microorganisms into a plurality of subcultures and subjecting the plurality of subcultures to a plurality of exaptation agents in order to obtain diversified substrains, wherein the exaptation agents are selected to favor survival of sub strains, or sets of sub strains, exhibiting an ability to outcompete the Multiple-Antibiotic Resistant Infection. The steps of dividing and subjecting may be optionally iterated, using at least some of the diversified substrains in place of the original population, until at least some ability to outcompete the Multiple-Antibiotic Resistant Infection is achieved. Performance of each of the diversified substrains is assessed with respect to ability to outcompete the Multiple-Antibiotic Resistant Infection, and those of the diversified substrains have the ability to outcompete the Multiple-Antibiotic Resistant Infection are selected. At least one kill switch is preferably incorporated into the antiMARI microorganism. The kill switch may be a requirement of the antiMARI for at least one component not available in nature, wherein the antiMARI is unable to adapt or exapt around the absence of the component. The component may be a synthetic nutrient that must be supplied to a patient or a non-canonical amino acid. The exaptation agents may include at least mutagenesis agents, training, horizontal gene transfer opportunities, and stressors. At least some of the subcultures may also be subjected to at least one adaptation-evoking agent. The invention incudes any treatment for a Multiple-Antibiotic Resistant Infection created according to the method.

### BRIEF DESCRIPTION OF THE DRAWINGS

Other aspects, advantages and novel features of the invention will become more apparent from the following detailed description of the invention when considered in conjunction with the accompanying drawings, wherein:

FIG. 1 is a flowchart of an example embodiment of a method for directed exaptation using diversification, according to one aspect of the invention.

FIG. 2 is flowchart of an example implementation of a method for directed exaptation resulting in the creation of the “United States of E. coli” (U.S.E.) multiculture, according to one aspect of the invention.

FIG. 3 is a schematic representation of a specific U.S.E. experiment, according to the example implementation of FIG. 2.

FIG. 4 depicts the 96 well plate chemical mutagen and UV mutagen treatments used in the experiment of FIG. 3.

FIG. 5 is a graph of experimental results from the experiment of FIG. 3, showing optical density versus time of growth for the original E. coli strain, U.S.E., and NEB Turbo competent E. coli populations.

FIG. 6 is a flowchart of an example implementation of a method for directed exaptation resulting in creation of a personalizable/customized antiMARI therapeutic, according to one aspect of the invention.

### DETAILED DESCRIPTION

In preferred embodiments, the method of the invention expands laboratory-based directed evolution via adaptation and direct genome engineering methods to include directed exaptation. Embodiments of the present invention comprise a novel, generalizable methodology for directing the process of exaptation in a variety of evolving systems, including bacterial populations, resulting in the creation of new functions when starting with an existing function in extant organisms.

In a preferred embodiment of a method for directed exaptation according to the invention, genetically and/or phenotypically diverse multicultures of microorganism populations are rapidly generated from a monoculture, including, but not limited to, microorganisms capable of leveraging horizontal gene transfer, in general and specifically, when presented with stressors. Both genetic and phenotype changes are within the scope of the invention, including cases where the genome remains the same but the transcriptome or epigenome changes, affecting the phenotype, and where the genome, transcriptome, and epigenome remain the same, yet the phenotype becomes different before any genomic changes are detectable. The invention includes a process for production of varying types of monoculture strains that maximize the probability for exaptation to occur at the individual or population level and may be used to rapidly screen for optimal performance under arbitrary challenge conditions.

As used herein, the following terms expressly include, but are not to be limited to:

“Adaptation” means an enhancement of an existent trait resulting in an increased fitness of an organism or population.

“Adaptation-evoking agent” means any mechanism that can be used to cause adaptation, causing an organism or population to change in order to become better suited to its environment by enhancement of an existent trait.

“Exaptation” means an evolving system's capacity to discover new functions by acquiring and deploying one or more adaptive traits in novel ways. The term exaptation as used herein also includes instances of individuals or populations carrying mutations to existent genes and/or acquiring genes via horizontal transfer resulting in mono- or multi-cultures of a species that outperform the ancestral strain at adaptation.

“Exaptation agent” means any mechanism that can be used to cause exaptation by causing a system to discover new functions by acquiring and deploying one or more adaptive traits in novel ways. Examples of exaptation agents for microorganisms include, but are not limited to, mutagenesis agents, training, horizontal gene transfer opportunities, and environmental and other stressors.

“Horizontal gene transfer” is synonymous with “lateral genetic information transfer” and means the incorporation of a (set of) whole gene(s) into an individual's genome as a result of ingesting (cannibalizing) the genetic material of another individual, as is known to happen in E. coli.

“Kill switch” means a component upon which the microorganisms of a culture are critically dependent for survival, which component is unavailable in nature, such that the microorganisms are also unable to readily adapt or exapt around the lack of the component. Examples include a human-controlled component that is unavailable in nature, such as, for instance, a synthetic nutrient that must be supplied externally to a patient or incorporating non-canonical amino acids into the microorganism.

“Monoculture” means a population of cells each carrying identical genotype.

“Multiculture” means a population of cells of the same species with individuals exhibiting genetic variability.

In preferred embodiments, a monoculture microorganism population is amplified if necessary and then divided into separate substrain populations having initially identical composition. The substrain populations are then diversified through any of the many means known in the art including, but not limited to, mutagenesis, training (exposure to various environmental conditions), horizontal gene transfer opportunities, and stressors. The processes of amplification, division, and diversification may be optionally repeated as many times as is desired or necessary, until a library of genetically and/or phenotypically diversified substrains has been created. The performance of individual diversified substrains or sets of pooled substrains are then assayed against each other and/or against various standards to identify substrains and/or pools of substrains that meet one or more of the desired criteria.

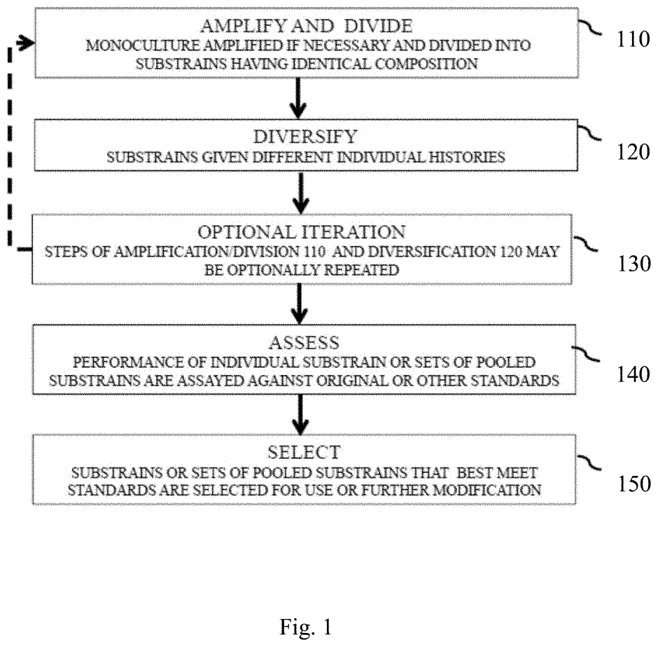

FIG. 1 is a high-level flowchart of key steps in an example generalized embodiment of the directed exaptation method of the invention. In FIG. 1, an “original”/“ancestral” monoculture is amplified if necessary and divided 110 into separate parts of identical composition (substrains). Substrains are then diversified 120 by giving them different individual histories through a variety of exaptation agents, such as, for example, but not limited to, mutagenesis, training, horizontal gene transfer opportunities, and environmental or other stressors. The steps of amplification/division 110 and diversification 120 may be optionally repeated 130 as many times as is desired or necessary, resulting in a library of diversified substrains. Performance of individual substrains or sets of pooled substrains is assessed 140 against each other and/or original or other standards. Substrains or sets of pooled substrains that best meet the assessment criteria are then selected 150 for use or for further modification.

“United States of E. coli” (U.S.E.) Application

In an example implementation of a method for directed exaptation according to the invention, a protocol was developed to create diverse populations from a starting monoculture of E. coli, with each subculture being subjected to a different combination of mutagenic stressors, shaping its own survival strategy. The subcultures were then combined, resulting in a “multicultural” E. coli that was shown to outcompete both the ancestral monoculture and a commercially available monoculture that was specifically genetically engineered to be “Fast growers” under the challenge condition.

In this example implementation, the “Original” (B39.1) monoculture strain was split into over 600 subcultures, and each was subjected to a unique combination of chemical and UV mutagenesis. These subcultures were grown at 18° C. (considered cold-shock treatment, which was the Evolthon challenge condition). This allows for internal competition within each subculture, with the survivors surrounded by the remnants of the less fortunate, with the overall stress adding to the propensity of lateral genetic information propagation (horizontal gene transfer). The double mutagenized (chemical+UV exposed) bacteria were additionally co-incubated with pasteurized “Fast” bacteria ((NEB turbo competent E. coli C298, heat killed and exposed to antibiotic they did not have resistance to) in order to potentially confer an adaptive advantage. Finally, double mutagenized (chemical+UV), single mutagenized (chemical or UV only), and Original bacteria, representing a total of 613 different subcultures, were pooled to create the “United States of E. coli” (U.S.E.) multiculture, which was shown to grow at 18° C. in the presence of bacteria from competing teams.

The method used to create U.S.E. was designed explicitly to yield a multiculture having maximum adaptability to unknown stressors. Directed evolution methods of E. coli, cultured under various stressors before being pooled into one culture, gain the ability to be in general better, i.e. faster and more resilient when faced with having to adapt to the unknown. Such a multiculture is expected to be overall more robust at surviving multiple types of challenge as compared to a homogenous population evolved or designed to do well in one particular challenge.

To rapidly create diverse populations amounting to diverse origin histories of “multicultural” E. coli, each subculture was subjected to a different combination of mutagenic stressors, shaping their own unique survival strategy. Weak members may die, but their genetic information is available for incorporation via horizontal gene transfer into the multiculture as a type of memory of prior harsh conditions. These subcultures are then combined to create a multiculture of strains, having a single “nationality” conferred by the shared “passport” present in all members of the superculture as the B39.1 barcode.

In one sense, instead of only “survival of the most efficient divider during cold shock” the invention may be viewed as “general adaptive strength through diversity.” Specifically, those substrains that are “weak” under one stressor have potentially enormous value in this system, as they may become “strong” when the stressor changes. In species capable of horizontal gene transfer, such as E. coli, even individual cells that have lysed may transfer their genetic information to the surviving culture as a type of distributed memory.

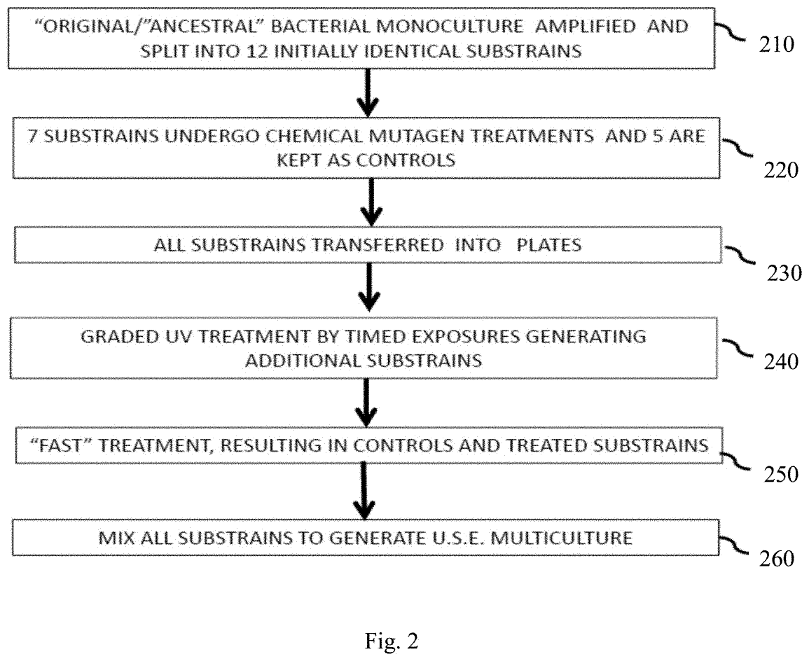

FIG. 2 is a high-level flow chart of steps comprising an example of implementation of directed exaptation resulting in the creation of the “United States of E. coli” (U.S.E.) multiculture, according to one aspect of the invention. As shown in FIG. 2, the “original/” ancestral” bacterial monoculture was amplified and split 210 into 12 initially identical substrains. Seven of the 12 initial substrains were subjected to chemical mutagen treatments 220, with 5 kept as controls. All substrains were grown at 18° C., applying the specific challenge condition of the Evolthon challenge 2016. All substrains were transferred 230 into 96 well plates and subjected to graded UV treatment 240 by timed exposures, generating additional substrains. Next, “Fast” treatment (NEB turbo competent E. coli C2984, pasteurized and treated with antibiotics before being added to media) was applied 250, resulting in 59 controls and 553 treated substrains. Finally, all 612 substrains +1 original substrain were mixed to generate the “United States of E. coli” (U.S.E.) multiculture, consisting of combinations of chemical and/or UV and/or “Fast” treated populations.

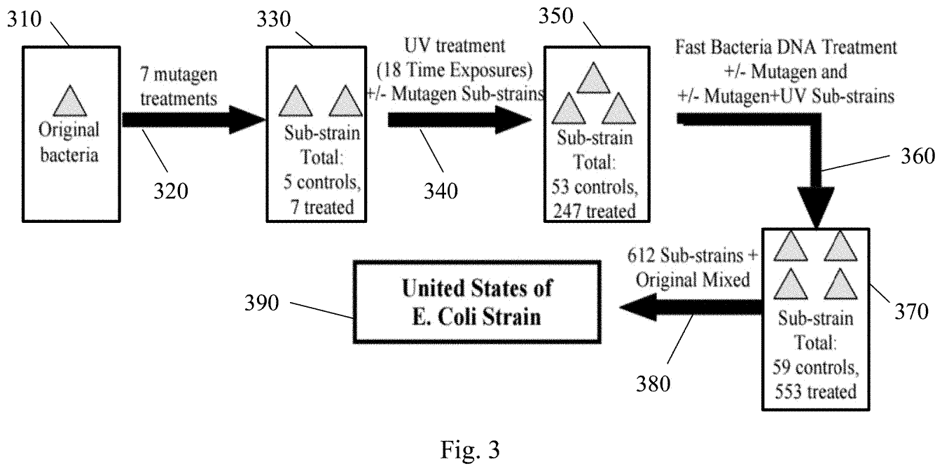

FIG. 3 is a schematic representation of a specific U.S.E. protocol, according to the example implementation of FIG. 2. As shown in FIG. 3, “Originals” (barcoded and titled “B39.1” by the Evolthon competition organizers) 310 were first chemically mutagenized 320, with each tube receiving a different treatment (or controls with no mutagen), then allowed to grow at 18° C. to select for adaptive mutations 330. The presumptive mutants and controls were then plated into 96 well plates and treated 340 with 18 differently graded doses of UV, resulting in 254 different subcultures 350, each exposed to a unique combination of chemical and UV mutagenesis. These subcultures were again allowed to grow at 18° C. The double mutagenized (chemical+UV exposed) bacteria were additionally co-incubated 360 with heat-killed “Fast” bacteria to potentially confer an adaptive advantage. Finally, double mutagenized (chemical+UV), single mutagenized (chemical or UV only), and Original bacteria, representing a total of 613 different subcultures 370, were pooled 380 to create the “United States of E. coli” (U.S.E.) culture 390.

Specific materials and methods for the creation of an implementation of U.S.E. follow.

Bacterial culture and strains: Evolthon E. coli strain number B39.1, a derivative of strain MG1655 with kanamycin resistance gene, is referred to as the “Original” or, synonomously, “Ancestral” monoculture strain. The Original strain of bacteria was inoculated in 50 mL of LB broth with 1:1000 kanamycin in a 125 mL flask and grown at 37° C., with shaking at 200 RPM, for 14 hrs. Stock of Originals was stored at 4° C. As mutant batches were created, their stocks were stored at 4° C. Long-term storage for genome sequencing was at −80° C. NEB Turbo competent E. coli (C2984), referred to as “Fast”, were used as a control and an additional DNA source. Fast bacteria used were not kanamycin resistant.

Materials: Phenol (Phen) (Sigma Aldrich P1037-25G), formaldehyde (PFA) (Sigma Aldrich F8775-25ML), potassium hydroxide (KOH) (Sigma Aldrich 221473-25G), copper sulfate (CuSO4) (Sigma Aldrich C1297-100G), ammonium chloride (NH4Cl) (Sigma Aldrich 326372-25G), hydrogen peroxide (H2O2) (Sigma Aldrich H3410-500ML), and caffeine (Caff) (Sigma Aldrich C0750-5G) were diluted in LB broth with kanamycin. A Mini Benchtop UV Transillumminator (UVP TM-10E), 6 W, 115V, 302 nm wavelength, was used for ultraviolet treatment.

Methods. Chemical Mutagenesis: Original stock culture as prepared above was diluted 1:1000 in LB and mutagens were added in 14-mL round-bottom Falcon tubes, with a single mutagen per tube (Corning 352059). Mutagen concentrations and exposure times were based on protocols from “A Survey of Chemicals for Mutagenic Action on E. coli,” by M. Demerec, G. Bertani and J. Flint, The American Naturalist Vol. 85, No. 821 (March-April, 1951), pp. 119-136. Treatment occurred at room temperature (RT). After treatment, mutagenized E. coli were spun down at 8,000 RPM, resuspended in 2 mL media and incubated first at RT for 24 h, recovered at 37 C for 24 h, then grown further at 18 C, prior to UV treatment. Mutagen concentrations, treatment duration, and post-treatment incubation times used for the example implementation of U.S.E. are listed in Table 1.

TABLE 1

| | | | Incubation |
|---|---|---|---|
| | | | Post- |
| | Concentration | Duration | Treatment |
| Treatment | (%) | (hrs) | (hrs) |
| Phen | 0.01% | 3.5 | 24 |
| PFA | 0.01% | 3.5 | 24 |
| KOH | 0.02% | 3.5 | 24 |
| CuSO₄ | 0.20% | 3.5 | 24 |
| NH₄Cl | 0.25% | 3.5 | 24 |
| H₂O₂ | 0.20% | 0.75 | 24 |
| Caff | 2.10% | 24 | 1 |

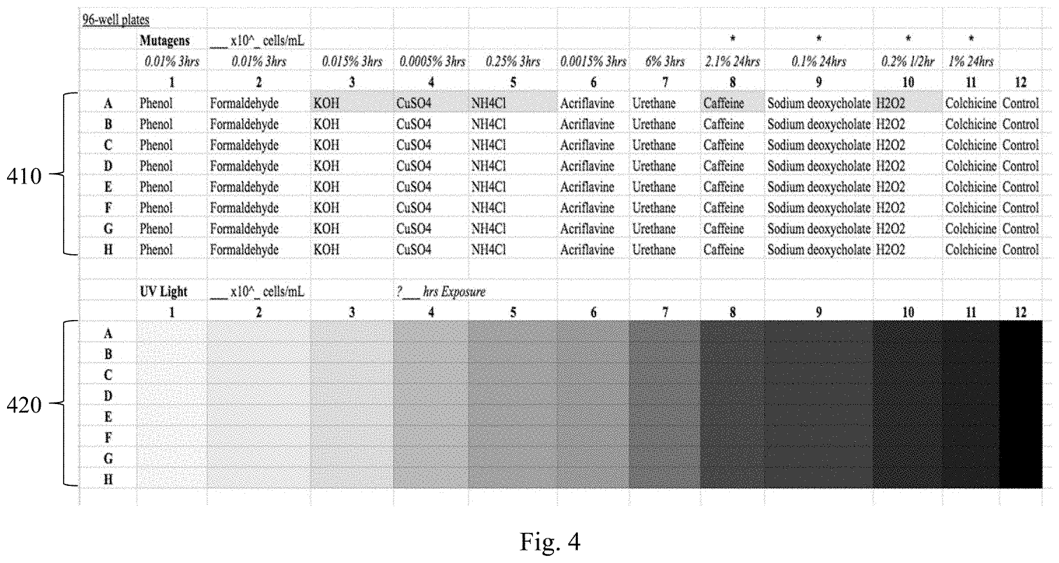

Ultraviolet Mutagenesis: Aliquots of each of the 7 mutagenized and 5 control subcultures were diluted 1:1000 in LB and treated in 250 ul in 1 mL 96-well plates (USA Scientific 1896-1110). Each plate had two controls (received no UV exposure), and treatment times were 10 seconds, 20 s, 40 s, 1 m, 1.5 m, 2 m, and every minute up to 14m (18 separate exposure times per chemically mutagenized subculture). UV Exposure was through the wells from the bottom at RT. After exposure, plates were incubated at 37° C. for 24 hrs then moved to 18° C. for 96 hrs. FIG. 4 depicts the 96 well plate chemical mutagen 410 and UV mutagen 420 treatments used in the experiment of FIG. 3.

“Fast” Bacteria Treatment: Fast bacteria were inoculated in LB broth, no antibiotics, and were incubated at 37° C. for 18 hrs. Bacteria were killed via pasteurization at 75° C. for 4.5 hrs before the addition of kanamycin to the dead/non-dividing culture. Dead Fast bacteria were added to each of the 254 mutagenized subcultures, the non-mutagenized and single mutagenized controls, and stock culture of the Originals. Treatment was done in 1 mL 96-well plates with 1:5 by volume (50 ul) dead Fast bacteria to (250 ul) experimental bacteria. The final dilution of all sub-cultures, controls, and Originals as mentioned above was 1:250. They were incubated at 37° C. for 23 hrs then moved to 20° C. for 23 hrs.

Creation of United States of E. coli Strain: 50 uL from each of the 612 mutagenized and Fast bacteria treated subcultures, each exposed to a unique combination of chemical, UV, and heat-killed bacteria, were pooled to create the final competition strain (“U.S.E.”). This strain was compared to the Original and Fast. U.S.E. and Original were diluted 1:50 in 250 uL media; Fast bacteria were inoculated from a frozen sample. The three cultures were incubated at 20° C. for 48 hrs and optical density measurements were taken every 2 hrs at 600 nm with a NanoDrop (Thermo Scientific ND-1000);

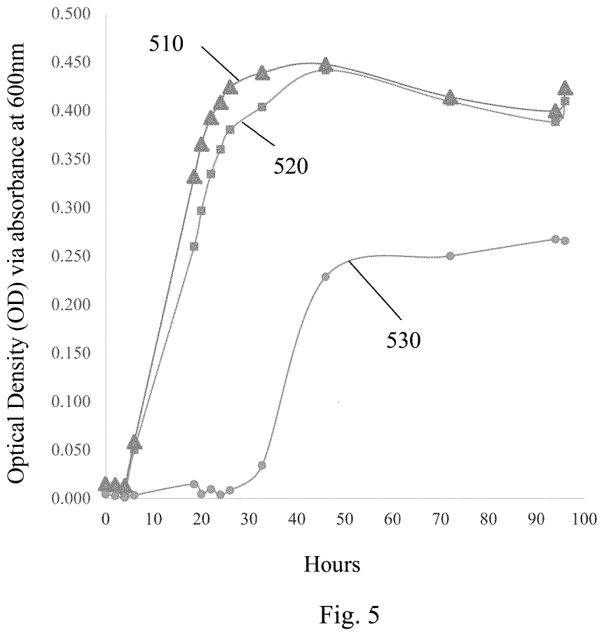

FIG. 5 is a graph of experimental results from the experiment of FIG. 3, showing optical density versus time of growth for the U.S.E. 510, original E. coli strain 520, and NEB Turbo competent E. coli (Fast) 530 populations in identical conditions over 100 hours. The curve is given in absolute units of units of absorbance (range 0-1), which is to be interpreted as Optical Density Units (OD) within arbitrary scaling factor. These data show that the “U.S.E.” multiculture 510 outperforms both the “Original” ancestral strain 520 and the “Fast” engineered E. coli 530.

AntiMARI Application

An example application of directed exaptation according to the present invention is the rapid prototyping of bacterial cultures developed to outcompete another population of bacteria, such as, for example, a Multiple-Antibiotic Resistant Infection (MARI) strain. In this implementation, the present invention is employed as a method for screening for optimal antiMARI activity. The selection of the most effective anti-MARI multiculture may be performed in high-throughput micro- and mesofluidic laboratory settings, as well as conventional Petri dishes, by comparing the effects of single or multiple antibiotics and other antibacterial treatments on a culture taken from a patient, versus a number of multicultures developed by the U.S.E. methodology described above. The end goal is to not just create anti-MARI treatments that out-consume the resources available to a MARI, eventually replacing its individuals with the antiMARI population, but also to optimize the selection given the constraints and efficacy of available treatments that may be either a) most effective at removing the antiMARI while ineffective against the original MARI or b) effective at suppressing the original MARI while having no effect against the anti-MARI.

To act as a therapeutic, once the antiMARI culture has dominated, it must be killed. This can be accomplished in some instances by screening the antiMARI for maximum susceptibility to one or more of the available antibiotic treatments which are (by definition in this case) ineffective against the original MARI. Especially in the cases where horizontal gene transfer is possible, this strategy caries significant risk of the antiMARI acquiring the same antibiotic resistance as the original MARI. For this, as well as for safety against fast-adapting/exapting potentially infectious microorganism populations escaping the laboratory, a generalizable “kill switch” strategy was developed.

“Kill switches” may be realized in a variety of ways sharing general attributes that involve controlling a necessary-for-survival component of the antiMARI that is unavailable in nature, such as, for instance, but not limited to, incorporating non-canonical amino acids [e.g. Hayden, Erika Check, “ ‘Radically rewritten’ bacterial genome unveiled', Nature, news, August 18, 2016, doi:10. 1038/nature.2016.20451]. The antiMARI cultures should be designed such that the anti-MARI are dependent for their survival on the availability of such component, while also unable to readily adapt or exapt around the lack of the component. The anti-MARI thus is preferably made critically dependent for its survival on the presence of a human-controlled component that is unavailable in nature (such as for instance a synthetic nutrient that must be supplied externally to the patient), and the anti-MARI is preferably unable to readily adapt or exapt around the lack of the critical component (such as for instance incorporating non-canonical amino acids).

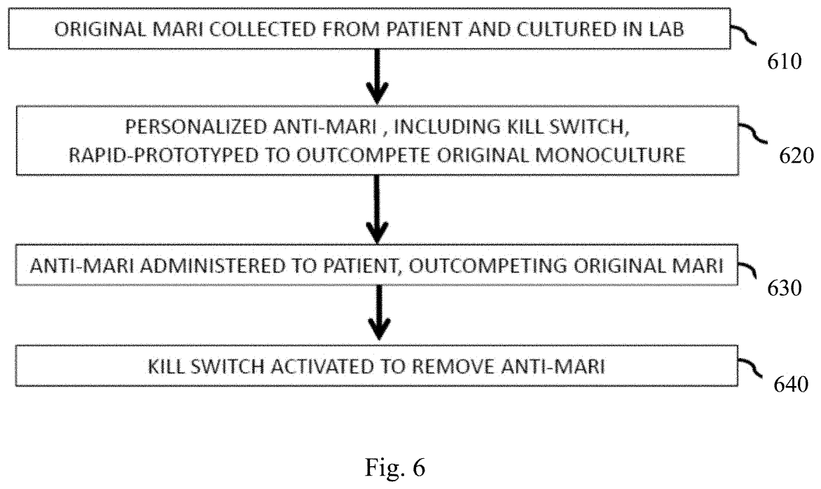

FIG. 6 is a high-level flowchart of an example implementation of a method for directed exaptation resulting in creation of a personalizable/customized antiMARI therapeutic, according to one aspect of the invention. As shown in FIG. 6, the original MARI is collected from the patient and cultured 610 in the lab. A personalized anti-MARI, having a Kill Switch, is rapid-prototyped 620 to outcompete the original MARI monoculture. The personalized anti-MARI is then administered 630 to the patient, whereupon it outcompetes the original MARI. Once the original MARI is gone, the Kill Switch is activated 640 to remove the antiMARI from the patient.

Biomanufacturing

Embodiments of the present invention comprise a novel modular, generalizable, design and methodology, which in exemplary embodiments may be applied in conjunction with external fluidics and automation/feedback loop control mechanisms, lowering the cost of explorative screenings. In example embodiments, the invention may employ fluidics designed for directed evolution via adaptation (such as those described in Mershin, Andreas, “Methods and apparatus for shifted-wavelength photosynthetic energy harvesting and biomass production”, U.S. Pat. App. Pub. No. 2017/0298312, Oct. 19, 2017; Mershin, Andreas, et al., “Methods and Apparatus for Transplantation of Nucleic Acid Molecules”, U.S. Pat. App. Pub. No. 2015/0037890, Feb. 5, 2015; Mershin, Andreas, et al., “Methods and Apparatus for Cell-Free Microfluidic- Assisted Biosynthesis”, U.S. Pat. App. Pub. No. 2016/0002611, Jan. 7, 2016; Lai, David, et al., “Methods and Apparatus for Variable Emulsification”, U.S. patent application Ser. No. 16/052,705, filed Aug. 2, 2018; and Mershin, Andreas, et al., “Synell Creation, Evolution, and Digital Transmission”, U.S. patent application Ser. No. 15/831,379, filed Dec. 4, 2017, all of which are herein incorporated by reference) and automation/feedback loop control mechanisms deployed via machine vision-enabled personal computer (PC), optionally in combination with variable substrate material properties, such as for example, but not limited to, polymer or peptide hydrogel 3D scaffolds (for example, tuned to simulate healthy or tumor tissue extracellular matrix).

Competition between cultures grown on traditional Petri dish or multi-well plates, as well as nano-, micro-, or meso- fluidic channels that can be observed via machine vision, leveraging automation and cell-confinement [Mershin, Andreas, et. al., “Methods and apparatus for transplantation of nucleic acid molecules”, PCT Pub. No. WO2015017733, Feb. 5, 2015], and automation-ready architectures are particularly suitable for metabolic engineering/biomanufacturing applications. A multiculture must be assayed as to relative fitness on various metrics of interest. This can be achieved using traditional methods, for instance, to quantify biosynthesis yields of small molecules using well-known Gas Chromatography-Mass Spectrometry (GC-MS), Liquid Chromatography-Mass Spectrometry (LC-MS), and Enzyme-Linked Immunosorbent Assay (ELISA), as well as by using less-well known machine olfaction high-throughput applications [e.g. U.S. Pat. No. 9,140,677]. Device geometry and appropriate tuning of flow rates, media nutrients and other physicochemical parameter (for example, but not limited to, temperature, pH, salinity, and dissolved gas concentrations) settings, in combination with material properties, such as, but not limited to, polymer or peptide hydrogel 3D scaffolds replicating healthy tissue extracellular matrix geometries, can be leveraged, providing high-throughput personalized screening of efficacy in real time.

In some embodiments of this method, instead of an optical or infrared (IR) microscope, a camera with an appropriate resolution at a specific field of view is interfaced with a smartphone or computer equipped with a computer vision library. In some embodiments, the camera is mounted orthogonally for number of cells detection and segmentation. Both image processing tasks are automated and optimized based on the ambient light conditions, allowing objective particle segmentation using image processing filtering algorithms that aim to extract the outline of the cells and quantify by morphology and number. Adding fluorescent or other labels to one or more substrains of specific genotypes allows for the multiplexed quantification and observation of subpopulation dynamics under varying conditions.

In example implementations of this invention, a feedback loop controls the levels of nutrients, stressors, and other physicochemical parameters in order to discover the optimal settings for ensuring that the resultant multiculture has the desired characteristics either in absolute terms, such as, for example, yield of a molecular product, or in relative terms, such as, for example, by real-time comparison to the performance of a stable control. In conjunction with inventions such as that taught in Lai, David, et al., “Methods and Apparatus for Variable Emulsification”, U.S. patent application Ser. No. 16/052,705, filed Aug. 2, 2018, this allows automated condition discovery in varying environments, agnostic to the actual nature of the materials being used.

Further, such optimization tasks when done in parallel with a control can be performed without explicitly tracking environmental variations that affect the system as a whole, such as, but not limited to, temperature or pressure, and do not need to be explicitly measured or monitored, because the feedback loop system only depends on the user's defined desired outcome. Embodiments of the invention therefore include one or more methods that can be integrated to implement biomanufacturing. The methods can be implemented under known paradigms, or may be integrated to implement new paradigms or approaches to synthetic biology and biomanufacturing.

In some implementations of this invention, multicultures can be assembled from previously prepared and frozen stocks of known genetic background. In some implementations of this invention, multicultures can be created and screened continuously, allowing stable scaleup continuous production. In some implementations, this invention is configured to discover new genotypes resulting in same phenotype. In some implementations, this invention is configured to study microbiome populations.

The present invention has application in the fields of basic research into directed evolution, genome engineering, and design. It provides a means to answer questions that are currently difficult to assess experimentally with statistical, generalizable significance and across different organisms, including under what conditions adaptive pressures cause exaptation, how adaptation and exaptation interact at the individual and population levels and whether known exaptations, such as multicellularity emergence, may be achieved in the laboratory.

This invention permits, for the first time, controllable directed evolution towards exaptation as well as adaptation. Outcomes are controlled by the experimenter through the results of basic IF/THEN logic operations responsive to global and/or local physicochemical parameters, including molecular species-specific concentrations, pH, temperature, illumination, salinity, and mutagenic and other factors. The ultimate generalizable impact of the methods of the invention is expansion of the number and efficacy of conditions screened in the search for directed evolutionary adaptive or exaptive outcomes, including for the creation of new functions starting from existing functions in extant organisms.

While preferred embodiments of the invention are disclosed herein, many other implementations will occur to one of ordinary skill in the art and are all within the scope of the invention. Each of the various embodiments described above may be combined with other described embodiments in order to provide multiple features. Furthermore, while the foregoing describes a number of separate embodiments of the apparatus and method of the present invention, what has been described herein is merely illustrative of the application of the principles of the present invention. Other arrangements, methods, modifications, and substitutions by one of ordinary skill in the art are therefore also considered to be within the scope of the present invention.

What is claimed is:

1. A method for creating a multiculture microorganism population exapted to meet at least one specific challenge condition, comprising the steps of:
dividing an original population comprising a monoculture of a plurality of the microorganisms into a plurality of subcultures having initially identical composition;
subjecting at least some of the plurality of subcultures to a plurality of different exaptation agents to obtain diversified substrains of the microorganism, wherein at least a first portion of the subcultures is subjected to different types or combinations of exaptation agents than are subcultures in other portions. and wherein at least one of the exaptation agents is selected to favor survival of substrains of the microorganism that meet the at least one specific challenge condition;
optionally iterating the steps of dividing and subjecting, using at least a subset of the diversified substrains in place of the original population, until a specified level of diversification of substrains is achieved; and
combining at least some of the diversified substrains with unexapted microorganisms from the original monoculture to create the multiculture microorganism population.

2. The method of claim 1, wherein all of the diversified substrains are combined with unexapted microorganisms from the original monoculture to create the multiculture microorganism population.

3. The method of claim 1, further comprising:
assessing the performance of each of the diversified substrains with respect to the at least one specific challenge condition; and
selecting only those of the diversified substrains that meet predetermined performance criteria for the at least one specific challenge condition for combination with the unexapted microorganisms from the original monoculture to form the multiculture microorganism population.

4. The method of claim 1, wherein at least one of the exaptation agents is selected from the group consisting of mutagenesis agents, training, horizontal gene transfer opportunities, and stressors.

5. The method of claim 1, further comprising the step of co-incubating at least a subset of the diversified substrains with at least one other living or dead microorganism condition before combining the diversified substrains with the unexapted microorganisms from the original monoculture to form the multiculture microorganism population, wherein the at least one other microorganism is known to be preferentially adapted to meet the specific challenge.

6. The method of claim 5, wherein at least one of the exaptation agents is selected from the group consisting of mutagenesis agents, training, horizontal gene transfer opportunities, and stressors.

7. The method of claim 1, wherein the step of subjecting further comprises the step of subjecting at least one portion of the plurality of subcultures to at least one adaptation-evoking agent.

8. The method of claim 1, further comprising the step of co-incubating at least a subset of the diversified substrains with at least one other living or dead microorganism condition before combining the diversified substrains with the unexapted microorganisms from the original monoculture to form the multiculture microorganism population, wherein the at least one other microorganism is known to be preferentially adapted to have an adaptive advantage of fast growth.

9. The method of claim 1, wherein the specific challenge condition is the ability to outcompete a Multiple-Antibiotic Resistant Infection, the exaptation agents are selected to favor survival of substrains exhibiting an ability to outcompete the Multiple-Antibiotic Resistant Infection, the specified level of diversification of substrains is the ability to outcompete the Multiple-Antibiotic Resistant Infection, and the predetermined performance criteria include the ability to outcompete the Multiple-Antibiotic Resistant Infection, such that the multiculture microorganism population is an antiMARI microorganism population.

10. A method for producing a treatment for a Multiple-Antibiotic Resistant Infection, comprising the steps of:
creating an antiMARI microorganism by the steps of :
dividing an original population comprising a monoculture of a plurality of microorganisms into a plurality of subcultures having initially identical composition;
subjecting at least some of the plurality of subcultures to a plurality of different exaptation agents to obtain diversified substrains of the microorganism, wherein the exaptation agents are selected to favor survival of sub strains, or sets of substrains, wherein at least a first portion of the subcultures is subjected to different types or combinations of exaptation agents than are subcultures in other portions, and wherein at least one of the exaptation agents is selected to favor survival of substrains of the microorganism that exhibiting an ability to outcompete the Multiple-Antibiotic Resistant Infection;
optionally iterating the steps of dividing and subjecting, using at least a subset of the diversified substrains in place of the original population, until an ability to outcompete the Multiple-Antibiotic Resistant Infection is achieved; and
combining at least some of the diversified substrains with microorganisms from the original monoculture to create the Multiple-Antibiotic Resistant Infection treatment.

11. The method of claim 10, further comprising the step of incorporating at least one kill switch into the antiMARI microorganism.

12. The method of claim 11, wherein the kill switch is a requirement of the antiMARI microorganism for at least one component not available in nature, wherein the antiMARI microorganism is unable to adapt or exapt around the absence of the component.

13. The method of claim 12, wherein the component is a synthetic nutrient that must be supplied to a patient or a non-canonical amino acid.

14. The method of claim 10, wherein all of the diversified substrains are combined with unexapted microorganisms from the original monoculture to create the Multiple-Antibiotic Resistant Infection treatment.

15. The method of claim 10, further comprising:
assessing the performance of each of the diversified substrains with respect to ability to outcompete the Multiple-Antibiotic Resistant Infection treatment; and
selecting only those of the diversified substrains that have the ability to outcompete the Multiple-Antibiotic Resistant Infection for combination with the microorganisms from the original monoculture to form the Multiple-Antibiotic Resistant Infection treatment.

16. The method of claim 10, wherein at least one of the exaptation agents is selected from the group consisting of mutagenesis agents, training, horizontal gene transfer opportunities, and stressors.

17. The method of claim 10, further comprising the step of co-incubating at least a subset of the diversified substrains with at least one other living or dead microorganism condition before combining the diversified substrains with the microorganisms from the original monoculture to form the Multiple-Antibiotic Resistant Infection treatment, wherein the at least one other microorganism is known to be preferentially adapted to outcompete the Multiple-Antibiotic Resistant Infection.

18. The method of claim 17, wherein at least one of the exaptation agents is selected from the group consisting of mutagenesis agents, training, horizontal gene transfer opportunities, and stressors.

19. The method of claim 10, wherein the step of subjecting further comprises the step of subjecting at least one portion of the plurality of subcultures to at least one adaptation-evoking agent.

20. The method of claim 10, further comprising the step of co-incubating at least a subset of the diversified substrains with at least one other living or dead microorganism condition before combining the diversified substrains with the unexapted microorganisms from the original monoculture to form the Multiple-Antibiotic Resistant Infection treatment, wherein the at least one other microorganism is known to be preferentially adapted to have an adaptive advantage of fast growth.
